(19) United States
(12) Patent Application Publication
(10) Pub. No.: US 2008/0226608 A1
(43) Pub. Date: Sep. 18, 2008

(54) Methods of Isolating and Using Enriched Subpopulations of Bone Marrow Progenitors for the Treatment of Diabetes

(75) Inventor: David A. Hess, London (CA)

Correspondence Address: SENNIGER POWERS LLP (WSHU), ONE METROPOLITAN SQUARE, 16TH FLOOR, SAINT LOUIS, MO, 63102, US

(73) Assignee: WASHINGTON UNIVERSITY, St. Louis, MO (US)

(21) Appl. No.: 12/014,521

(22) Filed: Jan. 15, 2008

Related U.S. Application Data
(60) Provisional application No. 60/884,943, filed on Jan. 15, 2007.

(51) Int. Cl. A61K 35/12; C12N 5/06; A61P 7/12; C12Q 1/32

(52) U.S. Cl. 424/93.7; 435/372; 435/26

(57) ABSTRACT

Enriched subpopulations of bone marrow progenitors are obtained according to methods that involve the staining of bone marrow cells and the identification of bone marrow progenitors within the stained cell population having specific intracellular fluorescence or orthogonal light scatter properties. Enriched subpopulations of the described progenitors may be used to lower blood glucose levels and treat hyperglycemia, and in particular hyperglycemia caused by Type 1 or Type 2 diabetes.

### CROSS REFERENCE TO RELATED APPLICATIONS

This application is a nonprovisional of U.S. patent application Ser. No. 60/884,943, filed Jan. 15, 2007, which is hereby incorporated herein by reference in its entirety.

### FIELD OF THE INVENTION

The present invention generally relates to compositions comprising bone marrow or bone marrow derived progenitors and methods of using the same for the prophylaxis or treatment of diabetes.

### BACKGROUND

The prevalence of diabetes is increasing at epidemic proportions. Although islet transplantation, combined with immunosuppressive therapy, can establish insulin independence in patients with type 1 diabetes, a severe shortage of donor tissue limits islet transplantation to less than 0.5% of the 1.3 million patients with severe type I diabetes in the US. Thus, novel sources of insulin-secreting cells and methods of using the same to establish insulin independence are needed.

### SUMMARY OF THE INVENTION

Among the various aspects of the present invention is the provision of preventing or treating diabetes using the compositions and methods disclosed herein.

Briefly, therefore, one aspect of the present invention is directed to a subpopulation of enriched bone marrow or bone marrow derived progenitors useful for the treatment or prophylaxis of diabetes. The subpopulation of progenitors exhibits an intracellular fluorescence that is at least about 2 times less than the fluorescence of at least about 90% of the population of bone marrow cells from which the subpopulation is enriched when the population is contacted with a fluorescently detectable substrate of ALDH.

Another aspect of the present invention is a method of treating a patient at risk for developing diabetes or having diabetes. The method comprises administering a therapeutic or prophylactic amount of a subpopulation of enriched progenitors as described herein to the patient.

Another aspect of the present invention is a method of lowering or maintaining lowered blood glucose levels in a patient suffering from or at a risk of suffering from elevated blood glucose levels. The method comprises administering a therapeutic or prophylactic amount of a subpopulation of enriched progenitors as described herein to the patient.

Yet another aspect of the claimed invention is a method of isolating a subpopulation of enriched bone marrow or bone marrow derived progenitors for use in the prophylaxis or treatment of diabetes. The method comprises exposing a population of bone marrow cells to a fluorescing substrate of aldehyde dehydrogenase (ALDH); and selecting a subpopulation of progenitors from the population of bone marrow cells, wherein said subpopulation displays an intracellular fluorescence less than the intracellular fluorescence of about 90% of the cells in said population of bone marrow cells and an orthogonal light scatter less than the orthogonal light scatter of about 90% of the cells in said population of bone marrow cells as determined by a measurement of intracellular fluorescence intensity and orthogonal light scatter.

Other objects and features will be in part apparent and in part pointed out hereinafter.

### BRIEF DESCRIPTION OF THE DRAWINGS

FIGS. 1A and 1B illustrate flow cytometric analyses of aldehyde dehydrogenase activity of cells from human umbilical cord blood (FIG. 1A) and human bone marrow (FIG. 1B) according to experiments described in Example 1.

FIGS. 2A and 2B are graphs illustrating the effect of ALDH^{hi} and ALDH^{lo} progenitors from human umbilical cord blood (FIG. 2A) and ALDH^{hi} and ALDH^{lo} progenitors from human bone marrow (FIG. 2B) on blood glucose concentrations in STZ treated NOD/SCID β2M null mice according to the experiments described in Example 1.

FIGS. 3A, 3B, and 3C are photos of human bone marrow ALDH^{hi} cells cultured in EGM-2 media (FIG. 3A) and secondarily cultured in MATRIGEL™ media subsequent to culturing in EGM-2 media (FIG. 3B) and ALDH^{lo} cells cultured in AMNIOMAX™ media (FIG. 3C) according to the experiments described in Example 3.

FIG. 4 is a graph illustrating the effects of transplantation of cultured human bone marrow ALDH^{lo} cells on glucose concentration in STZ treated NOD/SCID β2M null mice having hyperglycemia according to the experiments described in Example 3.

### DETAILED DESCRIPTION OF THE INVENTION

Surprisingly, it has been discovered that a particular subpopulation of bone marrow or bone marrow derived progenitors are useful in the prophylaxis or treatment of diabetes, both Type 1 and Type 2, and in particular Type 1 diabetes. This particular subpopulation of cells may generally be referred to herein as bone marrow or bone marrow derived aldehyde dehydrogenase low (ALDH^{lo}) progenitors or progenitor cells. Certain of these progenitors may also exhibit a low side scatter (sometimes referred to herein as low orthogonal light scatter), in which case this particular subpopulation may be referred to herein as bone marrow or bone marrow derived aldehyde dehydrogenase low, side scatter low (ALDH^{lo}SSC^{lo}) progenitors or progenitor cells. Certain of these progenitors may also be mesenchymal cells. In cultured populations of the enriched subpopulations of progenitors, the cultured populations may comprise exclusively or almost exclusively mesenchymal cells, in which case the cells may sometimes be referred to herein as bone marrow or bone marrow derived mesenchymal progenitors or bone marrow or bone marrow derived aldehyde dehydrogenase low, side scatter low (ALDH^{lo}SSC^{lo}) mesenchymal progenitors. Typically, this enriched subpopulation of cells exhibits a low expression of aldehyde dehydrogenase and, therefore, exhibits a low intracellular fluorescence relative to other cells in the bone marrow population from which the subpopulation is enriched as measured by fluorescence activated cell sorting subsequent to contact with an aldehyde dehydrogenase substrate. This subpopulation of cells may also exhibit a low side scatter relative to the side scatter of the other cells in the bone marrow population from which the subpopulation is enriched as measured by the same methods.

Enriched Subpopulation of Progenitors

Generally, the subpopulation of enriched bone marrow or bone marrow derived progenitors may exhibit a low internal fluorescence, in particular as measured by fluorescence activated cell sorting (FACS), relative to the internal fluorescence of other cells in the bone marrow population from which the subpopulation is enriched when contacted with an aldehyde dehydrogenase (ALDH) substrate. The cells of the enriched subpopulation may exhibit, for example, an internal fluorescence of less than about 10⁴ units, preferably less than about 10³ units, more preferably less than about 10² units, still more preferably less than about 10¹ unit, even more preferably between about 10⁰ and about 10² units, and most preferably between about 10¹ and about 10² units. Typically, the cells of the enriched subpopulation exhibiting the described internal fluorescence characteristics will account for less than about 50% of the cells, preferably less than about 60%, 70%, 80%, 90%, 92%, 95%, 97%, and 99% of the cells of the bone marrow or bone marrow derived cell population from which they are enriched. Accordingly, by way of example, the embodiments of enriched subpopulations of progenitors listed in Table 1 may be achieved.

TABLE 1
Enriched Subpopulations of Progenitors Having a Particular
Internal Fluorescence

| | Number of Enriched |
|---|---|
| | Progenitors, as a Percentage |
| | of the Total Bone Marrow/Bone |
| | Marrow Derived Cell |
| | Population, Having a Particular |
| Internal Fluorescence | Fluorescence |
| Less than about 10³ units | Not more than about 50% |
| Less than about 10³ units | Not more than about 40% |
| Less than about 10³ units | Not more than about 30% |
| Less than about 10³ units | Not more than about 20% |
| Less than about 10² units | Not more than about 50% |
| Less than about 10² units | Not more than about 40% |
| Less than about 10² units | Not more than about 30% |
| Less than about 10² units | Not more than about 20% |
| Less than about 10¹ units | Not more than about 40% |
| Less than about 10¹ units | Not more than about 30% |
| Less than about 10¹ units | Not more than about 20% |
| Less than about 10¹ units | Not more than about 10% |
| Less than about 10¹ units | Not more than about 8% |
| Less than about 10¹ units | Not more than about 5% |
| Between about 10⁰ and about 10² units | Not more than about 50% |
| Between about 10⁰ and about 10² units | Not more than about 40% |
| Between about 10⁰ and about 10² units | Not more than about 30% |
| Between about 10¹ and about 10² units | Not more than about 50% |
| Between about 10¹ and about 10² units | Not more than about 40% |
| Between about 10¹ and about 10² units | Not more than about 30% |
| Between about 10¹ and about 10² units | Not more than about 20% |
| Between about 10¹ and about 10² units | Not more than about 10% |
| Between about 10¹ and about 10² units | Not more than about 8% |

Accordingly, in one embodiment, the subpopulation of enriched bone marrow or bone marrow derived progenitors exhibit an intracellular fluorescence less than 10² units when contacted with a fluorescently detectable substrate of ALDH and comprises not more than about 10% of the population of bone marrow cells from which the subpopulation is enriched. In another embodiment, the subpopulation of enriched bone marrow or bone marrow derived progenitors exhibit an intracellular fluorescence less than 10² units when contacted with a fluorescently detectable substrate of ALDH and comprises not more than about 8% of the population of bone marrow cells from which the subpopulation is enriched. In yet another embodiment, the subpopulation of enriched bone marrow or bone marrow derived progenitors exhibit an intracellular fluorescence less than 10² units when contacted with a fluorescently detectable substrate of ALDH and comprises not more than about 5% of the population of bone marrow cells from which the subpopulation is enriched. In yet another embodiment, the subpopulation of enriched bone marrow or bone marrow derived progenitors exhibit an intracellular fluorescence of between about 10⁰ to about 10² units when contacted with a fluorescently detectable substrate of ALDH and comprises not more than about 10% of the population of bone marrow cells from which the subpopulation is enriched. In still another embodiment, the subpopulation of enriched bone marrow or bone marrow derived progenitors exhibit an intracellular fluorescence of between about 10⁰ to about 10² units when contacted with a fluorescently detectable substrate of ALDH and comprises not more than about 8% of the population of bone marrow cells from which the subpopulation is enriched. In another embodiment, the subpopulation of enriched bone marrow or bone marrow derived progenitors exhibit an intracellular fluorescence of between about 10⁰ to about 10² units when contacted with a fluorescently detectable substrate of ALDH and comprises not more than about 5% of the population of bone marrow cells from which the subpopulation is enriched. In another embodiment, the subpopulation of enriched bone marrow or bone marrow derived progenitors exhibit an intracellular fluorescence of between about 10¹ to about 10² units when contacted with a fluorescently detectable substrate of ALDH and comprises not more than about 10% of the population of bone marrow cells from which the subpopulation is enriched. In still another embodiment, the subpopulation of enriched bone marrow or bone marrow derived progenitors exhibit an intracellular fluorescence of between about 10¹ to about 10² units when contacted with a fluorescently detectable substrate of ALDH and comprises not more than about 8% of the population of bone marrow cells from which the subpopulation is enriched. In still another embodiment, the subpopulation of enriched bone marrow or bone marrow derived progenitors exhibit an intracellular fluorescence of between about 10¹ to about 10² units when contacted with a fluorescently detectable substrate of ALDH and comprises not more than about 5% of the population of bone marrow cells from which the subpopulation is enriched.

Generally, the cells of the enriched subpopulation exhibiting the described internal fluorescence may also have an orthogonal light scatter or side scatter that is less than the orthogonal light scatter or side scatter of a majority of the bone marrow cells from which the subpopulation is enriched. Typically, the orthogonal light scatter or side scatter of the progenitors of the enriched subpopulation is less than the orthogonal light scatter or side scatter of about 50%, 60%, 70%, 80%, 90%, 92%, 95%, 97%, and 99% of the population of bone marrow cells from which the subpopulation is enriched. Accordingly, by way of example, the embodiments of enriched subpopulations of progenitors listed in Table 2 may be achieved.

TABLE 2
Enriched Subpopulations of Progenitors Having a Particular
Internal Fluorescence and a Particular Side Scatter

| | | Number of Enriched |
|---|---|---|
| | Number of Enriched | Progenitors, as a |
| | Progenitors, as a | Percentage of the Total |
| | Percentage of the | Bone Marrow/Bone |
| | Total Bone | Marrow Derived Cell |
| | Marrow/Bone | Population, Having a |
| | Marrow Derived Cell | Particular Fluorescence |
| Internal | Population, Having a | and a Particular Side |
| Fluorescence | Low Side Scatter | Scatter |
| Less than about 10³ | Less than about 50% | Not more than about 50% |
| units | | |
| Less than about 10³ | Less than about 40% | Not more than about 40% |
| units | | |
| Less than about 10³ | Less than about 30% | Not more than about 30% |
| units | | |
| Less than about 10² | Less than about 50% | Not more than about 50% |
| units | | |
| Less than about 10² | Less than about 40% | Not more than about 40% |
| units | | |
| Less than about 10² | Less than about 30% | Not more than about 30% |
| units | | |
| Less than about 10² | Less than about 20% | Not more than about 20% |
| units | | |
| Less than about 10¹ | Less than about 40% | Not more than about 40% |
| units | | |
| Less than about 10¹ | Less than about 30% | Not more than about 30% |
| units | | |
| Less than about 10¹ | Less than about 20% | Not more than about 20% |
| units | | |
| Less than about 10¹ | Less than about 10% | Not more than about 10% |
| units | | |
| Less than about 10¹ | Less than about 8% | Not more than about 8% |
| units | | |
| Less than about 10¹ | Less than about 5% | Not more than about 5% |
| units | | |
| Between about 10⁰ | Less than about 50% | Not more than about 50% |
| and about 10² units | | |
| Between about 10⁰ | Less than about 40% | Not more than about 40% |
| and about 10² units | | |
| Between about 10⁰ | Less than about 30% | Not more than about 30% |
| and about 10² units | | |
| Between about 10¹ | Less than about 50% | Not more than about 50% |
| and about 10² units | | |
| Between about 10¹ | Less than about 40% | Not more than about 40% |
| and about 10² units | | |
| Between about 10¹ | Less than about 30% | Not more than about 30% |
| and about 10² units | | |
| Between about 10¹ | Less than about 20% | Not more than about 20% |
| and about 10² units | | |
| Between about 10¹ | Less than about 10% | Not more than about 10% |
| and about 10² units | | |
| Between about 10¹ | Less than about 8% | Not more than about 8% |
| and about 10² units | | |

The subpopulation of enriched ALDH^{lo} bone marrow or bone marrow derived progenitors may also exhibit, for example, an internal fluorescence that is less than the internal fluorescence of a majority of the population of bone marrow or bone marrow derived cells from which the subpopulation is enriched when the population is contacted with a fluorescently detectable substrate of ALDH. For example, the progenitors may exhibit an internal fluorescence that is at least about two times less, at least about three times less, at least about four time less, at least about five times less, at least about ten times less, at least about twenty times less, at least about fifty times less, or at least about 100 times less than the fluorescence of at least about 50%, 60%, 70%, 80%, 90%, 92%, 95%, 97%, or 99% of the population of cells from which the subpopulation is enriched when the population is contacted with a fluorescently detectable substrate of ALDH. Accordingly, by way of example, the embodiments of enriched subpopulations of progenitors listed in Table 3 may be achieved.

TABLE 3
Enriched Subpopulations of Progenitors Having a Particular
Internal Fluorescence

| Internal Fluorescence of an | Number of Enriched Progenitors, as |
|---|---|
| Enriched Progenitors | a Percentage of the Total Bone |
| Relative to the | Marrow/Bone Marrow Derived Cell |
| Total Bone Marrow/Bone Marrow | Population, Having a Particular |
| Derived Cell Population | Fluorescence |
| Two times less | Not more than about 50% |
| Two times less | Not more than about 40% |
| Two times less | Not more than about 30% |
| Two times less | Not more than about 20% |
| Two times less | Not more than about 10% |
| Three times less | Not more than about 50% |
| Three times less | Not more than about 40% |
| Three times less | Not more than about 30% |
| Three times less | Not more than about 20% |
| Three times less | Not more than about 10% |
| Four times less | Not more than about 30% |
| Four times less | Not more than about 20% |
| Four times less | Not more than about 10% |
| Four times less | Not more than about 8% |
| Five times less | Not more than about 30% |
| Five times less | Not more than about 20% |
| Five times less | Not more than about 10% |
| Five times less | Not more than about 8% |
| Ten times less | Not more than about 20% |
| Ten times less | Not more than about 10% |
| Ten times less | Not more than about 8% |
| Ten times less | Not more than about 5% |
| Ten times less | Not more than about 3% |
| Ten times less | Not more than about 1% |

Accordingly, in one embodiment, the enriched bone marrow or bone marrow derived progenitors exhibit an internal fluorescence that is at least about two times less than about 50% of the population of bone marrow cells from which the subpopulation is enriched. In another embodiment, the enriched bone marrow or bone marrow derived progenitors exhibit an internal fluorescence that is at least about two times less than about 85% of the population of bone marrow cells from which the subpopulation is enriched. In another embodiment, the enriched bone marrow or bone marrow derived progenitors exhibit an internal fluorescence that is at least about two times less than about 90% of the population of bone marrow cells from which the subpopulation is enriched. In yet another embodiment, the enriched bone marrow or bone marrow derived progenitors exhibit an internal fluorescence that is at least about two times less than about 95% of the population of bone marrow cells from which the subpopulation is enriched.

Regardless of the manner in which the enriched subpopulation is defined based upon internal fluorescence, another aspect of the present invention is a composition comprising an enriched subpopulation of bone marrow or bone marrow derived ALDH^{lo}SSC^{lo} progenitors as described herein wherein the composition is about 90%, about 95%, about 97%, about 99% or about 100% bone marrow or bone marrow derived ALDH^{lo}SSC^{lo} progenitors. Generally, the bone marrow or bone marrow derived ALDH^{lo} or ALDH^{lo}SSC^{lo} progenitors can comprise a percentage of mesenchymal cells. Generally, the percentage of mesenchymal cells may be less than about 20%, 15%, 10%, 8%, 5%, or 1% of the ALDH^{lo} or ALDH^{lo}SSC^{lo} enriched subpopulations. These enriched subpopulations may be used in the compositions and formulations or in accordance with the methods disclosed herein. Alternatively, the enriched subpopulations of ALDH^{lo} or ALDH^{lo}SSC^{lo} progenitors may be cultured in vitro to select for specific progenitors, and in particular for mesenchymal progenitors, to thereby create compositions and formulations as described herein comprising at least about 50%, at least about 60%, at least about 70%, at least about 80%, at least about 90%, at least about 95%, at least about 97%, at least about 99% or about 100% mesenchymal progenitors. Enriched populations of mesenchymal cells may be cultured and selected for using media and methods known to those skilled in the art and suitable for the growth and maintenance of such cells, such as, for example, AMNIOMAX™ C-100 and AMNIOMAX™-II Complete media (Gibco/lnvitrogen, Carlsbad, Calif.). Briefly, with AMNIOMAX™ C-100 (a two part medium comprising a basal and a supplement component), the frozen supplement, when ready to be used, is completely thawed at 4° C. and gently swirled to ensure a homogeneous liquid. The entire content of the supplement is aseptically transferred to the AMNIOMAX™ C-100 basal and gently swirled to ensure a homogeneous complete medium. With the AMNIOMAX™-II Complete, the frozen medium, when ready to be used, is completely thawed at 4° C. and gently swirled to ensure a homogeneous liquid. Cells may then be grown in either medium according to methods known to those skilled in the art.

Formulations of the Compositions for Administration to Patients

The compositions described herein are suitable for use in the prophylaxis or treatment of hyperglycemia, and in particular hyperglycemia caused by or associated with diabetes, such as, for example, Type 1 diabetes. The compositions, therefore, can be prepared as formulations for administration to mammals, and in particular humans. Accordingly, the compositions may also comprise additional components that are typically used to formulate compositions suitable for administration to mammals. The addition of such components will depend in large part upon the mode of administration. The route of administration, and in particular the preferred route of administration, may depend on factors such as, for example, the disorder being treated and the severity of the disorder; activity of the specific compound employed; the specific composition employed; the age, body weight, general health, sex and diet of the patient; the time of administration; the rate of excretion of the specific compound employed; the duration of the treatment; drugs used in combination or coincidental with the specific compound employed and like factors well known in the medical arts. In any event, the composition may additionally comprise a “pharmaceutical carrier” such as a pharmaceutically acceptable buffer, suspending agent or vehicle for delivering the progenitor containing composition of the present invention to the mammal or human. The carrier may be liquid or solid, although it is preferably a liquid, and is selected consistent with the manner of administration. A “pharmaceutically acceptable” component is one that is suitable for use with humans and/or animals without undue adverse side effects (such as toxicity, irritation, and allergic response) commensurate with a reasonable benefit/risk ratio. Such components may be easily determined by those skilled in the art.

Formulations suitable for parenteral administration include aqueous and non-aqueous sterile formulations isotonic with the blood of the intended recipient; and aqueous and non-aqueous sterile suspensions which may include suspending systems designed to target the compound to blood components or one or more organs. The formulations may be presented in unit-dose or multi-dose sealed containers, for example, ampoules or vials. Injections, solutions and suspensions may be prepared from sterile powders, granules and tablets as known to those skilled in the art. Parenteral and intravenous forms may also include minerals or other materials to make them compatible with the type of injection or delivery system chosen.

In general, water, suitable oil, saline, aqueous dextrose (glucose), or related sugar solutions and glycols such as propylene glycol or polyethylene glycols are suitable carriers for parenteral solutions. Solutions for parenteral administration preferably contain a water soluble salt of the active ingredient, a carrier, and optionally stabilizing agents, buffers, or preservatives. Sodium bisulfite, sodium sulfite, ascorbic acid, citric acid or salts thereof, and EDTA or salts thereof are suitable stabilizing agents. Preservatives include, for example, benzalkonium chloride, methyl- or propyl-paraben, or chlorobutanol.

The present invention additionally contemplates administering compounds as described for use in the form of veterinary formulations, which may be prepared, for example, by methods that are conventional in the art in light of the present disclosure.

Methods of Enriching Bone Marrow for ALDH_{LO}SSC_{LO} Mesenchymal Progenitors

The present invention is also directed to a method of isolating an enriched subpopulation of bone marrow progenitors useful for the prophylaxis or treatment of hyperglycemia in a patient, and in particular, hyperglycemia typically caused by or associated with the onset or existence of diabetes in a patient. The method generally comprises exposing a population of bone marrow cells to a fluorescing substrate of aldehyde dehydrogenase (ALDH) and selecting a subpopulation of progenitors from the population of bone marrow cells, wherein said subpopulation displays an intracellular fluorescence less than the intracellular fluorescence of a majority of the ALDH-expressing cells in said population of bone marrow cells and an orthogonal light scatter less than the orthogonal light scatter of a majority of the ALDH-expressing cells in said population of bone marrow cells as determined by a measurement of intracellular fluorescence intensity and orthogonal light scatter.

Any number of fluorescing substrates of ALDH or precursors or analogs of fluorescing substrates may be used and are known to those skilled in the art. Generally, the fluorescing substrate, precursor, or analog thereof comprises a substrate bound to a fluorescent dye or stain, each of which is generally non-toxic to the cells and capable of entering or being transported into the cells and remaining therein for a period of time sufficient to allow the examination of the cells for internal fluorescence and orthogonal light scatter. A number of ALDH substrates are known to those skilled in the art and include, for example, aminoacetylaldehyde and precursors and analogs of the same. The substrate may be combined with (i.e., bound to or bound by) any number of fluorescent dyes to form fluorescing substrates, or precursors or analogs thereof, including, for example, dansylaminoacetaldehyde (DAAA) and Bodyipy™-aminoacetaldehyde (BAAA). In a particular embodiment, the fluorescing substrate, precursor, or analog thereof of ALDH is BAAA. This fluorescing substrate is manufactured and marketed as a component of a kit used for the staining and examination of bone marrow and bone marrow derived cells under the trade name Aldefluor® (Aldagen, Inc., Durham, N.C.).

Bone marrow cells and progenitors enriched from the same may be obtained from a number of sources and in manners well known to those skilled in the art. Sources for bone marrow or bone marrow cells include, for example, the tibia, the femur, the ulna, and the iliac crest. The bone marrow or bone marrow cells may be from any animal, including mammals and preferably humans. The bone marrow cells may also be lineage depleted according to methods known to those skilled in the art, such as, for example, the methods disclosed in Hess et al., Blood, 104(6): 16481656 (2004) and Hess et al., Blood, 107(5): 2162-2169 (2006), thereby decreasing the number of differentiated cells contained in the bone marrow cell population. The bone marrow or cells therefrom may be recently obtained (e.g., obtained from a patient or donor shortly before being subject to the methods disclosed herein) or stored (e.g., obtained from a patient or donor days to weeks before being subject to the methods disclosed herein), and may be fresh, frozen-thawed, or previously refrigerated.

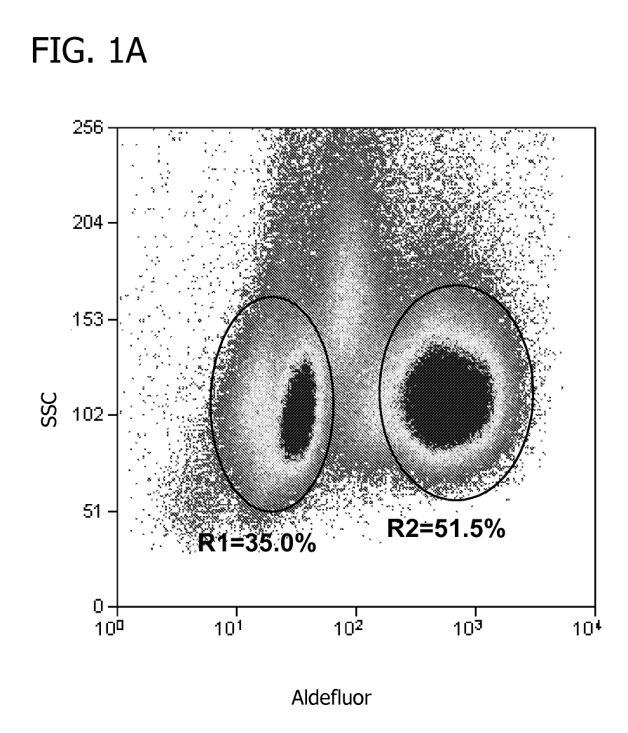

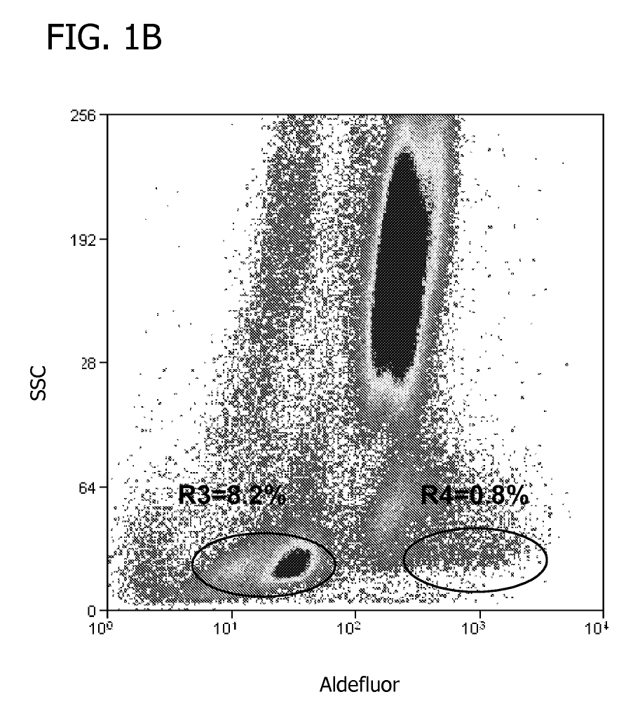

The population of bone marrow cells from which the subpopulation is enriched may be combined with and stained by the fluorescing substrate, precursor, or analog thereof in any manner known to those skilled in the art. Typically, the population of bone marrow cells may be incubated with the same for a period of about 1 to 60 minutes prior to being subjected to the methods of determining internal fluorescence and orthogonal light scatter of the cells contained therein. Preferably, the protocol is one that is consistent with the use of the Aldefluor® kit, with slight modifications to identify and isolate the ALDH^{lo}SSC^{lo} progenitors. Briefly, control and test samples of a bone marrow or bone marrow derived cell sample are created. An aliquot of diethylaminobenzaldehyde (DEAB) is added to the control sample test tube and an aliquot of DMSO-activated Aldefluor® substrate is added to the test sample test tube. An aliquot of the test sample is then immediately combined with the control sample in the control sample test tube. The test and control samples are then incubated for a period of about 30 to 60 minutes at about 37° C. After incubation, the samples are centrifuged, and the supernatants are removed. The test and control pellets are suspended in an aliquot of the Aldefluor® Assay Buffer. Samples may be analyzed immediately or cooled on ice or in a refrigerator and remain stable for 24 hours at a temperature of about 2° C. to about 8° C. The selected flow cytometer is the set up according to the manufacturer's instructions. An acquisition template is prepared by creating a forward scatter (FSC) vs. a side scatter (SSC) dot plot, creating a region (designated, for example, as R1) that will encompass the nucleated cells based on scatter. A fluorescence channel 1 (FL1) vs. SSC dot plot is then created gated on R1. In set-up mode, a DEAB control sample is placed on the flow cytometer. The FSC and SSC voltages and gains are adjusted to center the nucleated cell population within the FSC vs. SSC plot. The R1 region is adjusted to encompass the nucleated cell population based on scatter. On the FL1 vs. SSC plot, the FL1 photo-multiplier tube voltage is adjusted so that the right edge of the stained population is placed at the second log decade on the dot plot. The control tube is removed and the corresponding ALDH test sample is placed on the cytometer. A region designated, for example, as R2 is created to encompass the cell population that is ALDH^{lo}SSCₗₒ. The test sample is removed. For data acquisition, the analyzer is removed from set-up mode and a number of events, typically about 100,000, in R1 for each ALDH and DEAB sample using the same instrument settings are collected. An FSC vs. SSC dot plot is created. An R1 region in that plot is created to encompass the nucleated cells based on side scatter. Two FL1 vs. SSC dot plots are created gated on R1. An ALDH test sample data file is opened. The R1 region in the FSC vs. SSC dot plot is adjusted to encompass the nucleated cell population. On the first FL1 vs. SSC dot plot, an R2 region is created to encompass the cell population that is ALDH^{lo}SSC^{lo}. On the second FL1 vs. SSC dot plot, the corresponding DEAB control data file is used to verify the placement of the R2 region on the ALDH sample. There should be few or no events in the R2 region of the control tube. Region statistics are then added to the plot and the percentage of cells gated in R2 represents the nucleated events (R1) that are ALDH^{lo}SSC^{lo}. This particular population (i.e., ALDH^{lo}SSC^{lo} progenitors) is represented in the present application, for example, in FIG. 1B, as region R3.

The bone marrow or bone marrow derived cells and fluorescing substrate, precursor, or analog thereof may also be combined and incubated with an inhibitor of a multidrug resistance (MDR) pump, such as for example, the ABC G2 pump, to further enhance the staining of the cells. Any of a number of well known MDR pump inhibitors may be used, including, for example, verapamil and analogs thereof, in a concentration sufficient to inhibit, for example, the ABC G2 MDR pump, such as for example, a concentration of 50 μM. Methods of using MDR pump inhibitors in conjunction with ALDH fluorescing substrates, precursors, or analogs thereof are well known to those skilled in the art, as demonstrated, for example, in Storms et al., PNAS, 96(16): 9118-9123 (1999) and in U.S. Pat. No. 6,627,759 (Smith et al.).

As discussed above, the enriched subpopulation of progenitors may generally have particular internal fluorescence and orthogonal light scatter (i.e., side scatter) characteristics that allow for them to be identified within and enriched from a larger population of bone marrow cells. As discussed, therefore, the enriched cells may typically be ALDH^{lo} cells and, therefore, exhibit an internal fluorescence that is less than the internal fluorescence of about 50% of the bone marrow cells (i.e., the cells of the population from which the subpopulation is enriched), preferably less than the internal fluorescence of about 60% of the bone marrow cells, more preferably less than the internal fluorescence of about 70% of the bone marrow cells, still more preferably less than the internal fluorescence of about 80% of the bone marrow cells, even more preferably less than the internal fluorescence of about 90% of the bone marrow cells, yet more preferably less than the internal fluorescence of about 92% of the bone marrow cells, still more preferably less than the internal fluorescence of about 95% of the bone marrow cells, even more preferably less than the internal fluorescence of about 97% of the bone marrow cells, and most preferably less than the internal fluorescence of about 99% of the bone marrow cells.

As discussed above, the progenitors of the enriched subpopulation may also be SSC^{lo} and, therefore, the orthogonal light scatter or side scatter of the progenitors of the enriched subpopulation may be less than the orthogonal light scatter or side scatter of about 50% of the bone marrow cells (i.e., the cells of the population from which the subpopulation is enriched), preferably less than the orthogonal light scatter or side scatter of about 60% of the bone marrow cells, more preferably less than the orthogonal light scatter or side scatter of about 70% of the bone marrow cells, still more preferably less than the orthogonal light scatter or side scatter of about 80% of the bone marrow cells, even more preferably less than the orthogonal light scatter or side scatter of about 90% of the bone marrow cells, yet more preferably less than the orthogonal light scatter or side scatter of about 92% of the bone marrow cells, still more preferably less than the orthogonal light scatter or side scatter of about 95% of the bone marrow cells, even more preferably less than the orthogonal light scatter or side scatter of about 97% of the bone marrow cells, and most preferably less than the orthogonal light scatter or side scatter of about 99% of the bone marrow cells.

As also discussed above, the enriched subpopulation of bone marrow or bone marrow derived progenitors may comprise ALDH^{lo} or ALDH^{lo}SSC^{lo} progenitors wherein the composition is about 90%, about 95%, about 97%, about 99% or about 100% bone marrow or bone marrow derived ALDH^{lo}, or ALDH^{lo}SSC^{lo} progenitors. Generally, the bone marrow or bone marrow derived, ALDH^{lo} or ALDH^{lo}SSC^{lo} progenitors may be predominately mesenchymal progenitors. Generally, the bone marrow or bone marrow derived ALDH^{lo} or ALDH^{lo}SSC^{lo} progenitors can comprise a percentage of mesenchymal cells. Generally, the percentage of mesenchymal cells may be less than about 20%, 15%, 10%, 8%, 5%, or 1% of the ALDH^{lo} or ALDH^{lo}SSC^{lo} enriched subpopulations. These enriched subpopulations may be used in the compositions and formulations or in accordance with the methods disclosed herein. Alternatively, the enriched subpopulations of ALDH^{lo} or ALDH^{lo}SSC^{lo} progenitors may be cultured in vitro to select for specific progenitors, and in particular for mesenchymal progenitors, to thereby create compositions and formulations as described herein comprising at least about 50%, at least about 60%, at least about 70%, at least about 80%, at least about 90%, at least about 95%, at least about 97%, at least about 99% or about 100% mesenchymal progenitors. Enriched populations of mesenchymal cells may be cultured and selected for as discussed in greater detail above.

Once an enriched subpopulation of bone marrow or bone marrow derived progenitors is isolated according to the methods disclosed herein, an additional subpopulation of enriched bone marrow or bone marrow derived progenitors having the same characteristics may be prepared by in vitro culturing of the isolated enriched subpopulation. Methods of culturing cells are known to those skilled in the art. An example of such a method is described in Example 3, wherein the bone marrow or bone marrow derived progenitors described herein and obtained according to methods described herein were efficiently grown on MSC supportive (AMNIOMAX™) media. Generally, cells are grown in a medium capable of supporting the growth and maintenance of the cells. Such media typically contain serum, a nutrient, growth factors, serum, and the like and are maintained at a particular pH, temperature, and atmospheric composition during the growth cycle.

Methods of Treatment

The present invention is also directed to methods of prophylaxis or treatment of hyperglycemia in a patient, and in particular, hyperglycemia typically caused by or associated with the onset or existence of Type 1 diabetes in a patient. It is also directed to methods of lowering or decreasing blood glucose levels, or maintaining lowered blood glucose levels in a patient suffering from or at a risk of suffering from elevated blood glucose levels, and in particular, patients suffering from elevated blood glucose levels caused by or associated with the onset or existence of diabetes, and in particular, Type 1 diabetes. The methods generally comprise administering a therapeutic or prophylactic amount of the ALDH^{lo} or ALDH^{lo}SSC^{lo} progenitors described above to a patient, and in particular to a patient having or at risk for the development of diabetes, such as Type 1 diabetes. The method reduces blood glucose levels, for example, from about 200, 250, 300, 350, 400, 450, or 500 mg/dl or more, to a blood glucose level within an acceptable range, for example, a fasting blood glucose of about 65 to about 150 mg/dl, or about 70 to about 120 mg/dl, and/or maintains such a fasting blood glucose level.

Patients suitable for the treatment described herein include patients presenting with or suffering from the symptoms of elevated blood glucose levels, hyperglycemia, or diabetes, including Type 1 and Type 2 diabetes, including those in the pre-type 1 and honeymoon (i.e., early) stages of the disease. Suitable patients also include those at risk for developing elevated blood glucose levels, hyperglycemia, or diabetes, in particular Type 1 diabetes, including patients that exhibit Type 1 indicators, such as for example, expression of autoantibodies such as GAD65, IA-2 or IAA in their blood. Suitable patients include any mammal having or at risk for the development of elevated blood glucose levels, hyperglycemia, or diabetes or presenting with the described symptoms, including, for example, human, canine, feline, equine, bovine, or porcine patients.

Typically, the patient may be suffering from elevated blood glucose levels, hyperglycemia, or diabetes as a result of inadequate beta cell function or the disruption of processes that are mediated by beta cells or beta cell function. This may result from an inadequate number of beta cells due to, for example, the loss or decrease in the number of beta cells as a result of, for example, beta cell death; a decrease in or lack of production of beta cells; or the loss of function of existing beta cells.

Prophylaxis or treatment may include administration of a prophylactic or therapeutically effective amount of the compositions of the present invention in a form described herein to a subject in need of treatment. The compositions of the present invention can be administered in any well known manner that results in the decrease of blood glucose levels in the patient or the decrease or loss of symptomology associated with hyperglycemia or diabetes. Suitable manners of administering the compositions of the present invention include, for example, parenteral administration, including, for example, intravenous, intraarterial, subcutaneous, intramuscular, intraorbital, intracapsular, intraspinal, intraperitoneal, or intrasternal administration. The compositions may be administered in any conventional manner available for use in conjunction with pharmaceuticals, either as individual prophylactics or therapeutic agents or in a combination of prophylactic or therapeutics.

As with the selection of dosages, the preferred route of administration will vary with a number of factors, such as, for example, the disorder being treated and the severity of the disorder; activity of the specific compound employed; the specific composition employed; the age, body weight, general health, sex and diet of the patient; the time of administration; the rate of excretion of the specific compound employed; the duration of the treatment; drugs used in combination or coincidental with the specific compound employed and like factors well known in the medical arts. For example, it is well within the skill of the art to administer doses in a particular manner initially, for example, to cause a rapid increase or “loading” of the compound or composition in the patient prior to the administration of the compound in an alternative or different manner, for example, to maintain a particular level or concentration of the compound or composition in the blood or tissues of the patient.

Regardless of the mode of administration, generally targeted administration of the compositions disclosed herein may not be necessary, particularly as disclosed in the examples. Accordingly, systemic administration as disclosed above is contemplated. Alternatively, the compositions disclosed herein may be targeted to a particular organ, such as, for example, the liver or the pancreas, by methods known to those skilled in the art.

In addition to the administration of the bone marrow progenitors obtained according to the processes described herein, the methods of treatment described herein may also comprise the administration of additional prophylactic or therapeutic compounds. By way of example, the methods may also comprise the administration of islet cells from the pancreas of the patient or pancreata of donors, and in particular from donor cadavers. One example of a process of administering islets from donors, and in particular donor cadavers, is the Edmonton Protocol. The Edmonton Protocol is well known to those skilled in the art, as demonstrated by the disclosure in Shapiro, Ryan, and Lakey, Clinical Islet Transplantation—State of the Art, Transplantation Proceedings, 33: 3502-3503 (2001); Ryan et al., Clinical Outcomes and Insulin Secretion After Islet Transplantation With the Edmonton Protocol, Diabetes, 50: 710-719 (2001); Ryan et al., Continued Insulin Reserve Provides Long-Term Glycemic Control, Diabetes, 51: 2148-2157 (2002); and Shapiro et al., International Trial of the Edmonton Protocol for Islet Transplantation, New England Journal of Medicine, 355(13):1318-1330 (2006). Briefly, the Edmonton Protocol is a multistep protocol that generally first involves the step of delivery of LIBERASE™ or a similar enzymatic compound to a donor cadaver pancreas. LIBERASE™ digests the pancreas tissue without digesting the islets. Thereafter, there are several successive steps for separating the islets from other cells in the pancreas. The separated islets are then transplanted into the portal vein of the liver of diabetic patient. New blood vessels form in the liver to support the islets. The insulin that the cells produce is absorbed into the blood stream through these surrounding vessels and distributed through the body to control glucose levels in the blood. Generally, Edmonton Protocol patients will thereafter be required to take immunosuppressants, such as sirolimus and tacrolimus. They may also take a monoclonal antibody drug, such as daclizumab, to minimize or prevent the rejection of the donor islets.

Accordingly, in another embodiment the method of treating a patient at risk for developing or having elevated blood glucose levels, hyperglycemia, or diabetes, and in particular Type 1 diabetes, comprises administering to the patient a prophylactic or therapeutic amount of the ALDH^{lo} or ALDH^{lo}SSC^{lo} progenitors or an enriched population of ALDH^{lo} or ALDH^{lo}SSC^{lo} mesenchymal progenitors as described above and a prophylactic or therapeutic amount of donor islet cells. The range of islet cells administered may be from about 10³ to about 10¹² cells, preferably from about 10³ to about 10⁹ cells, more preferably from about 10³ to about 10⁶ cells, and most preferably about 10⁶ cells. The donor islet cells may be administered prior to, concurrently with, or subsequent to the administration of the ALDH^{lo} or ALDH^{lo}SSC^{lo} progenitors. Moreover, the islet cells may be administered with the ALDH^{lo} or ALDH^{lo}SSC^{lo} progenitors, either as a single dose comprising both components or as individual, multiple doses.

The methods of treatment described herein may also comprise administration of compositions capable of inhibiting the function of sensory neurons involved in the autoimmune reactions involved in diabetes. Specifically, the function of transient receptor potential vanilloid-1 (TRPV1) protein is believed to be involved in a feedback system that regulates β cell physiology within an optimal range (Razavi et al., Cell, 127(6): 1123-1135 (2006)). When this feedback system becomes unbalanced, particularly as a result of the defective function of TRPV1, pathogenesis of Type 1 diabetes is detectable. Administration of compounds that remove TRPV1+ neurons, such as, for example, capsaicin, or that disrupt the function of TRPV1+ neurons, such as, for example, neuropeptides like substance P, may eliminate the pathogenic interaction or transiently normalize the interaction, respectively, and thereby prevent or eliminate the Type 1 diabetic pathogenesis or symptomology.

Accordingly, in another embodiment the method of treating a patient at risk for developing diabetes or having diabetes, and in particular Type 1 diabetes, comprises administering to the patient a prophylactic or therapeutic amount of the ALDH^{lo} or ALDH^{lo}SSC^{lo} progenitors as described above and a prophylactic or therapeutic amount of a compound that eliminates TRPV1+ neurons or that normalizes the function of the same. In one embodiment, the compound eliminates TRPV1+ neurons. In a particular embodiment, the compound that eliminates TRPV1+ neurons is capsaicin. In another embodiment, the compound normalizes the function of TRPV1+ neurons. In a particular embodiment, the compound that normalizes the function of TRPV1+ neurons is substance P. These compounds may be administered prior to, concurrently with, or subsequent to the administration of the ALDH^{lo} or ALDH^{lo}SSC^{lo} progenitors. Moreover, the compounds may be administered with the ALDH^{lo} or ALDH^{lo}SSC^{lo} progenitors, either as a single dose comprising both components or as multiple individual doses.

Dosages of the Present Compositions

Any suitable dosage may be administered in the methods of the present invention. The form of the composition chosen for a particular application, the carrier, and the amount will vary widely depending on the species of the warm blooded animal or human, the disease or state thereof being treated, and the effective concentrations observed in trial studies.

The specific prophylactically or therapeutically effective dose level for any particular patient will depend upon a variety of factors including the disorder being treated and the severity of the disorder; activity of the specific compound employed; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of the compound at levels lower than those required to achieve the desired prophylactic or therapeutic effect and to gradually increase the dosage until the desired effect is achieved. If desired, the effective daily, weekly, or monthly dose may be divided into multiple doses for purposes of administration. Consequently, single dose compositions may contain such amounts or submultiples thereof to make up the daily, weekly, monthly, yearly or other dose.

Generally, a dose of least about 1×10³ cells per kilogram of body weight is administered to a patient depending on the factors mentioned above. Preferably, the dosage range of cells administered may be between about 1×10³ and about 5×10¹⁵ cells per kg of body weight, more preferably about 1×10³ to about 5×10¹² cells per kilogram of body weight, even preferably about 1×10³ to about 5×10⁹ cells per kilogram of body weight, still more preferably about 1×10³ to about 5×10⁶ cells per kilogram of body weight, and yet more preferably about 1×10³ to about 1×10⁶ cells per kilogram of body weight. In one embodiment, the dose is at about 1×10⁶ cells per kilogram of body weight.

The requisite dosage may be administered as a single administration of cells or multiple administrations of cells. Multiple administrations may be provided over the course of a single day, multiple days, weeks, or months. In some embodiments, multiple administrations are provided over the course of one to seven consecutive days. In some embodiments, three to seven administrations are provided over the course of three to seven days, and in particular consecutive days. In some embodiments, five administrations are provided over the course of five days, and in particular consecutive days.

In one embodiment, a single administration of between about 1×10³ and about 1×10¹⁵ cells per kilogram of body weight is administered. In another embodiment, a single administration of between about 1.5×10⁸ and about 1.5×10¹² cells per kilogram of body weight is administered. In another embodiment, a single administration of between about 1×10⁹ and about 5×10¹¹ cells per kilogram of body weight is administered. In another embodiment, a single administration of about 5×10¹⁰ cells per kilogram of body weight is administered. In yet another embodiment, a single administration of 1×10¹⁰ cells per kilogram of body weight is administered.

Generally the composition of the present invention may be administered on a daily, weekly, monthly or as needed basis, either in a single administration or separate administrations comprising one or more doses of the composition. Accordingly, in one embodiment, multiple administrations of about 1×10⁵ to about 1×10¹³ cells per kilogram of body weight are provided. In another embodiment, multiple administrations of about 1.5×10⁸ to about 1.5×10¹² cells per kilogram of body weight are provided. In another embodiment, multiple administrations of about 1×10⁹ to about 5×10¹¹ cells per kilogram of body weight are provided over the course of one to seven days, and preferably consecutive days. In another embodiment, multiple administrations of about 4×10⁹ cells per kilogram of body weight are provided over the course of one to seven days, and preferably consecutive days. In yet another embodiment, multiple administrations of about 2×10¹¹ cells per kilogram of body weight are administered over the course of one to seven days, and preferably consecutive days. In still another embodiment, five administrations of about 3.5×10⁹ cells are provided over the course of five days, and preferably consecutive days. In another embodiment, five administrations of about 4×10⁹ cells are provided over the course of five days, and preferably consecutive days. In another embodiment, five administrations of about 1.3×10¹¹ cells are provided over the course of five days, and preferably consecutive days. In another embodiment, five administrations of about 2×10¹¹ cells are provided over the course of five days, preferably consecutive days.

The dosage and the dosage regimen will vary depending on the ability of the patient to sustain the desired effective results. The dosing regime, therefore, may be a single dose administered once, once a day, once a week, once a month, biannually, annually, or even less frequently for the duration of the presentation of the patient's symptoms, for a period exceeding the same, or for the patient's lifetime either to treat or prevent the recurrence of symptomology. Likewise, the dosing regime may be multiple doses administered over the course of a single day, days, weeks, months, or years for the duration of the presentation of the patient's symptoms, for a period exceeding the same, or for the patient's lifetime either to treat or prevent the recurrence of symptomology.

Having described the invention in detail, it will be apparent that modifications and variations are possible without departing from the scope of the invention defined in the appended claims. Furthermore, it should be appreciated that all examples in the present disclosure are provided as non-limiting examples. Publications cited throughout this document are hereby incorporated herein by reference in their entirety.

### EXAMPLES

The following non-limiting examples are provided to further illustrate the present invention. It should be appreciated by those skilled in the art that the techniques disclosed in the examples that follow represent approaches the inventors have found function well in the practice of the invention, and thus can be considered to constitute examples of modes for its practice. However, those skilled in the art should, in light of the present disclosure, appreciate that many changes can be made in the specific embodiments that are disclosed and still obtain a like or similar result without departing from the spirit and scope of the invention.

### Example 1

The primary objective was to establish whether purified progenitor cells isolated from human umbilical cord blood (UCB) or adult bone marrow (BM) could induce functional recovery from hyperglycemia after transplantation into streptozotocin (STZ)-treated recipients. Experiments were 42-day trials with STZ-injections from days 1-5 to induce hyperglycemia, and cell transplantation by intravenous (i.v.) injection on day 10 after sublethal irradiation (300 cGy) in non-obese diabetic/severe combined immune deficient (NOD/SCID) β2 microglobullin (β2M) null mice. β-glucuronidase (GUSB) deficient NOD/SCID mucopolysaccharidosis type VII (MPS VII) mice were used for some experiments to facilitate the sensitive detection of human cell engraftment by GUSB staining.

Induction of hyperglycemia, blood glucose monitoring, BrdU injections. 8-10 week old immune deficient NOD/SCID mice (Jackson Laboratories, Bar Harbor, Me.) were intraperitoneally (IP) injected with 30 mg/kg STZ (Sigma-Aldrich., St. Louis, Mo.) daily for days 1-5. STZ was solubilized in citrate buffer pH 4.5 and injected within 15 minutes of preparation. Blood glucose was measured between 8-10 am, twice weekly from days 0-14 and weekly from days 14-49 with a glucometer Elite® diabetes care system (Bayer, AG, Germany). Peripheral blood (100 μL) was collected on days 0, 10, and 42, and serum insulin was quantified using a ¹²⁵I-labelled murine insulin-specific radio-immuno assay (Linco, St. Charles, Mo.). For the in vivo detection of proliferating cells in the pancreas of recipient NOD/SCID mice, Bromodeoxyuridine (5-bromo-2-deoxyuridine or BrdU) (50 μg/kg body weight in phosphate buffered saline (PBS) (Sigma-Aldrich, St. Louis, Mo.)) was IP injected 16 and 2 hours prior to pancreas extraction. NOD/SCID mice were maintained under sterile conditions in micro-isolator cages in ventilated racks using treatment and transplantation protocols approved by local animal care and ethics committees of the Robarts Research Institute and the University of Western Ontario.

Isolation of ALDH^{lo} and ALDH^{hi} cells. ALDH fractionation provides a non-toxic, reproducible isolation of progenitors based on a conserved stem cell function rather than cell surface markers (Hess et al, Blood, 104(6): 1648-1655, 2004). Cell populations were isolated by fluorescence activated cell sorting (FACS) as illustrated in FIG. 1 for lineage depleted (Lin⁻) UCB (FIG. 1A) or unfractionated human BM (FIG. 1B). Lineage depleted cells were purified for low (FIG. 1A, R1) or high (FIG. 1A, R2) ALDH activity. ALDH^{lo} and ALDH^{hi} cells represented approximately 35% and 50% of the UCB Lin⁻ population, respectively. BM mononucleated cells (MNCs) were also purified for low (FIG. 1B, R3) and high (FIG. 1B, R4) ALDH activity. ALDH^{lo} and ALDH^{hi} cells in human BM were approximately 8% and 0.8% of BM MNCs, respectively.

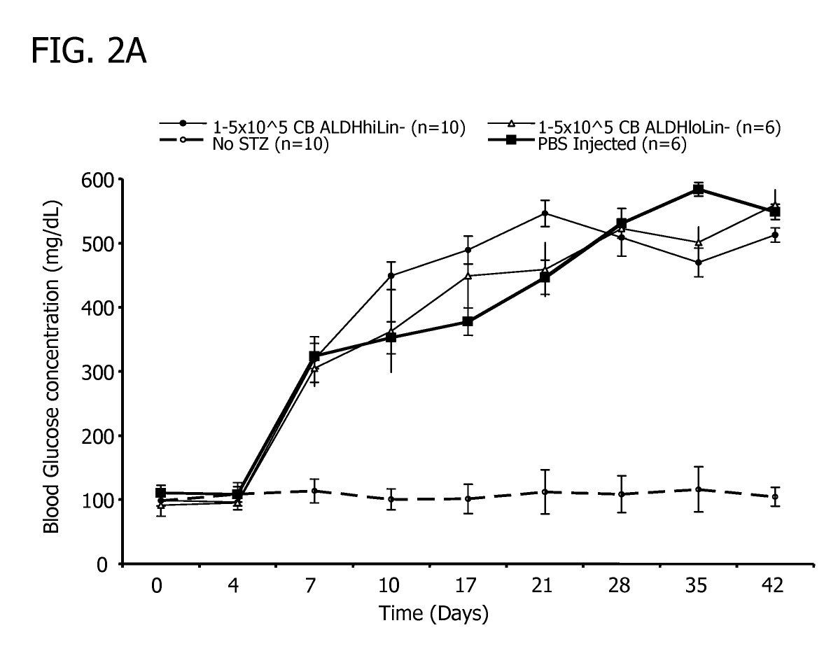

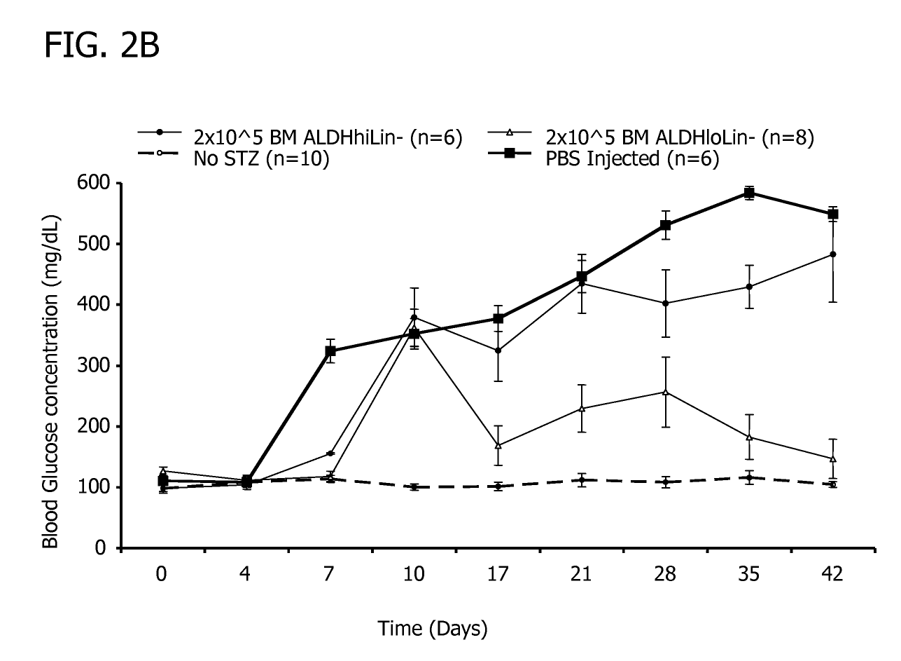

Transplantation of ALDHₗₒ and ALDHₕᵢ cells into hyperglycemic mice. Purified ALDH^{lo} and ALDH^{hi} cells from human UCB and human BM were transplanted by tail vein injection into sub-lethally irradiated (300 cGy) STZ-treated or vehicle-treated NOD/SCID β2M null mice. STZ-treated NOD/SCID β2M null mice were i.v. transplanted at day 10 with human UCB or BM-derived ALDH^{hi} or ALDH^{lo} cells and blood glucose concentration was monitored weekly. Transplantation of human UCB-derived ALDH^{hi}Lin⁻ (line with solid circles) or ALDH^{lo}Lin⁻ (line with hollow triangles) cells did not reduce blood glucose concentrations compared to PBS-injected controls (FIG. 2A). In contrast, i.v. injection of ALDH^{lo} cells but not ALDH^{hi} cells from human BM induced a significant reduction (*p<0.05) in blood glucose concentration (FIG. 2B), which was maintained for up to 30 days post-transplantation. Thus, it appears that the human BM-derived ALDH^{lo} population contains cells implicated in the regeneration of beta cell function after STZ-treatment. Surprisingly, the frequency of human cells in the pancreata of transplanted mice was very low as determined by flow cytometry (<0.5% human CD45+/human HLA A,B,C+). Therefore homing studies in STZ-treated NOD/SCID MPSVII mice were performed to investigate the interaction between transplanted human cells and regenerating recipient insulin-producing P-cells.

### Example 2

EPC and MSC culture from human BM. It has been previously shown that human ALDH^{hi} cells are a rich source of primitive, repopulating hematopoietic cells, but contain few progenitors from other lineages (Hess et al, Blood 104(6): 1648-1655, 2004). In contrast, BM is a heterogeneous mixture of mature and primitive cells containing repopulating hematopoietic progenitor cells (HPC), pro-angiogenic endothelial progenitor cells (EPC), and supportive mesenchymal stem cells (MSC). Because recovery from hyperglycemia was only observed after the transplantation of BM cells, it was hypothesized that mesenchymal or endothelial progenitors contained within the BM ALDH^{lo} population mediated the reduction of blood glucose.

Flow cytometric analysis for cell surface proteins normally expressed on hematopoietic and/or endothelial progenitors, revealed that BM ALDH^{hi} cells had increased expression of primitive hematopoietic markers CD34 (p<0.05) and c-kit (p<0.01), compared to BM ALDH^{lo} cells, as well as increased expression of CD133 (p<0.05), a primitive progenitor marker expressed in hematopoietic, endothelial and neural systems. Nearly all ALDH^{hi} cells expressed platelet derived endothelial cell adhesion molecule (PECAM-1), however, the mature endothelial cell marker VE-cadherin was not expressed. These data suggest that the BM-derived ALDH^{hi} cells are highly enriched for primitive hematopoietic and endothelial progenitors, compared to ALDH^{lo} cells.

### Example 3

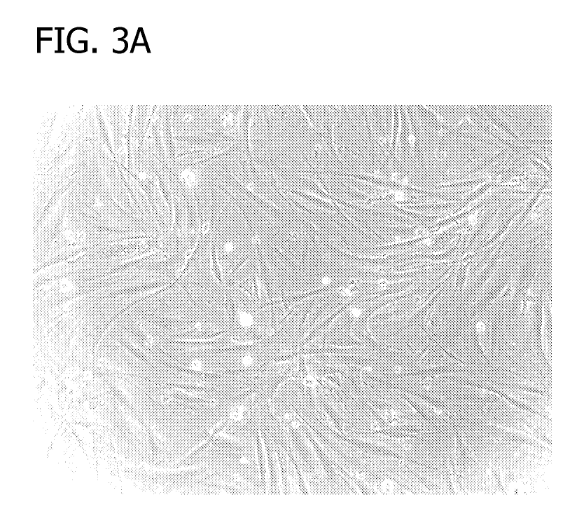

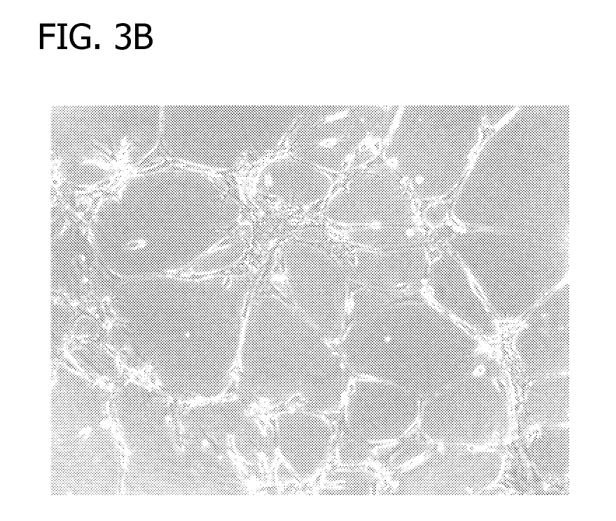

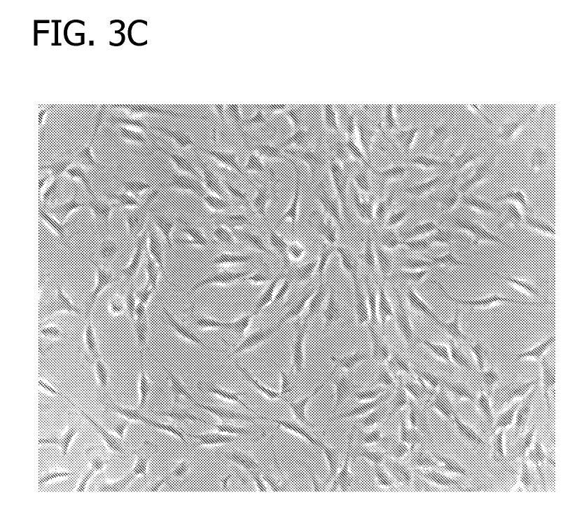

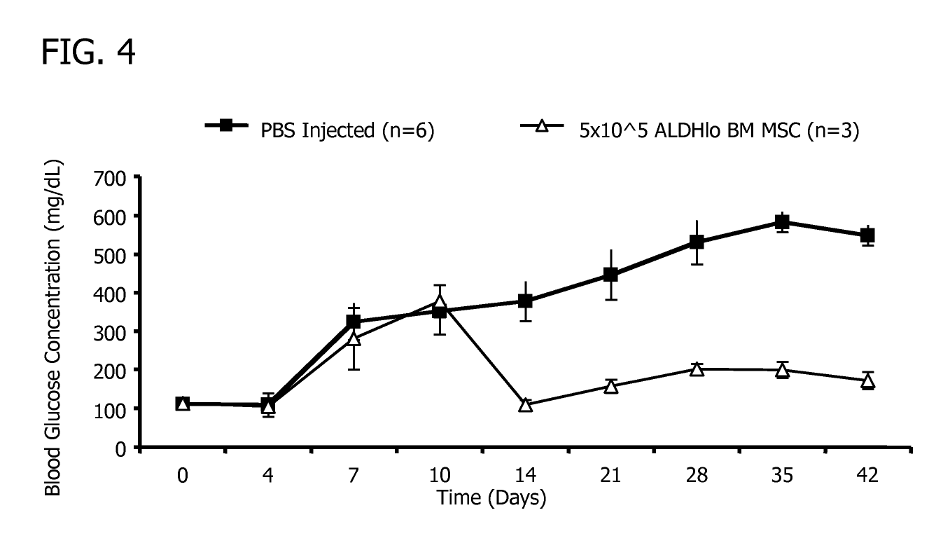

Correlation of phenotype with function. To correlate phenotype with function in vitro, we cultured human BM-derived ALDH^{lo} and ALDH^{hi} cells under conditions that promote the outgrowth of either HPC or EPC or MSC (FIG. 3). BM ALDH^{hi} cells were cultured for 28 days in endothelial cell media supplemented with EGF, IGF, bFGF, and VEGF and subsequently replated on MATRIGEL™. As predicted by phenotypic analysis, HPC function in methylcellulose was restricted to BM-derived ALDH^{hi} cells with the formation of distinct erythroid, macrophage, and granulocyte colonies (data not shown). Likewise, culture of BM-derived ALDH^{hi} cells, but not ALDH^{lo} cells in EPC media with pro-angiogenic growth factors resulted in the outgrowth of colonies with endothelial cell spindle shape morphology (FIG. 3A). The endothelial phenotype of these cultured cells was confirmed by flow cytometry (CD45−/CD144+), and secondary culture of these cells on MATRIGEL™ media resulted in tubule formation (FIG. 3B). Conversely, human BM-derived ALDH^{lo} cells efficiently grew MSC-like cells in MSC supportive media (AMNIOMAX™ media) (FIG. 3C). These MSC-like cells could be expanded in vitro for less than 10 passages without losing the capacity to produce adipose or bone in differentiation cultures (data not shown). Thus, human BM ALDH^{hi} cells possessed robust HPC and EPC function, whereas, ALDH^{lo} cells were enriched for MSC function, suggesting ALDH^{lo} MSC may be the cellular mediators of the regeneration of endogenous β-cell function after transplantation. Transplantation of cultured human MSC derived from BM ALDH^{lo} cells (expanded for 7 days in culture) into STZ-treated hyperglycemic mice effectively reduced hyperglycemia compared to PBS injected controls (FIG. 4) and did so for at least 28 days post-transplantation.

What is claimed is:

1. A subpopulation of enriched bone marrow or bone marrow derived progenitors useful for the treatment or prophylaxis of diabetes, said subpopulation of progenitors exhibiting an intracellular fluorescence that is at least about 2 times less than the fluorescence of at least about 90% of the population of bone marrow cells from which the subpopulation is enriched when the population is contacted with a fluorescently detectable substrate of ALDH.

2. The subpopulation of claim 1, wherein the bone marrow or bone marrow derived progenitors are adult human bone marrow or bone marrow progenitors

3. The subpopulation of claim 1, wherein the subpopulation of progenitors has an orthogonal light scatter less than the orthogonal light scatter of about 80% of the population of bone marrow cells from which the subpopulation is enriched.

4. The subpopulation of claim 1, wherein the subpopulation of progenitors has an orthogonal light scatter less than the orthogonal light scatter of about 90% of the population of bone marrow cells from which the subpopulation is enriched.

5. The subpopulation of claim 1, wherein the subpopulation of progenitors has an orthogonal light scatter less than the orthogonal light scatter of about 92% of the population of bone marrow cells from which the subpopulation is enriched.

6. The subpopulation of claim 1, wherein the subpopulation of progenitors has an orthogonal light scatter less than the orthogonal light scatter of about 95% of the population of bone marrow cells from which the subpopulation is enriched.

7. The subpopulation of claim 1, wherein the subpopulation of progenitors has an orthogonal light scatter less than the orthogonal light scatter of about 97% of the population of bone marrow cells from which the subpopulation is enriched.

8. The subpopulation of claim 1, wherein the subpopulation is enriched for mesenchymal progenitors.

9. The subpopulation of claim 8, wherein the subpopulation of enriched mesenchymal progenitors comprises at least about 80% mesenchymal progenitors.

10. The subpopulation of claim 8, wherein the subpopulation of enriched mesenchymal progenitors comprises at least about 90% mesenchymal progenitors.

11. The subpopulation of claim 8, wherein the subpopulation of enriched mesenchymal progenitors comprises at least about 95% mesenchymal progenitors.

12. A method of isolating a subpopulation of enriched bone marrow or bone marrow derived progenitors for use in the prophylaxis or treatment of diabetes, said method comprising:
exposing a population of bone marrow cells to a fluorescing substrate of aldehyde dehydrogenase (ALDH); and
selecting a subpopulation of progenitors from the population of bone marrow cells, wherein said subpopulation displays an intracellular fluorescence less than the intracellular fluorescence of about 90% of the cells in said population of bone marrow cells and an orthogonal light scatter less than the orthogonal light scatter of about 90% of the cells in said population of bone marrow cells as determined by a measurement of intracellular fluorescence intensity and orthogonal light scatter.

13. The method of claim 12, wherein the bone marrow or bone marrow derived progenitors are adult human bone marrow or bone marrow derived progenitors.

14. The method of claim 12, wherein the subpopulation displays an intracellular fluorescence less than the intracellular fluorescence of about 92% of the cells in said population of bone marrow cells.

15. The method of claim 12, wherein the subpopulation displays an intracellular fluorescence less than the intracellular fluorescence of about 95% of the cells in said population of bone marrow cells.

16. The method of claim 12, wherein the subpopulation displays an intracellular fluorescence less than the intracellular fluorescence of about 97% of the cells in said population of bone marrow cells.

17. The method of claim 12, wherein the subpopulation displays an orthogonal light scatter less than the orthogonal light scatter of about 92% of the cells in said population of bone marrow cells.

18. The method of claim 12, wherein the subpopulation displays an orthogonal light scatter less than the orthogonal light scatter of about 95% of the cells in said population of bone marrow cells.

19. The method of claim 12, wherein the subpopulation displays an orthogonal light scatter less than the orthogonal light scatter of about 97% of the cells in said population of bone marrow cells.

20. The method of claim 12, the method further comprising the step of culturing the subpopulation of enriched progenitors.

21. The method of claim 20, wherein the cultured subpopulation of enriched progenitors comprises at least about 80% mesenchymal progenitors.

22. The method of claim 20, wherein the cultured subpopulation of enriched progenitors comprises at least about 90% mesenchymal progenitors.

23. The method of claim 20, wherein the cultured subpopulation of progenitors is at least about 95% mesenchymal progenitors.

24. A method of lowering or maintaining lowered blood glucose levels in a patient suffering from or at a risk of suffering from elevated blood glucose levels, said method comprising administering a therapeutic or prophylactic amount of a subpopulation of enriched progenitors of claim 1 to the patient.

25. The method of claim 24, wherein the patient is a human.

26. The method of claim 24, wherein the therapeutic or prophylactic amount of the subpopulation of enriched progenitors is about 1×10³ and about 1×10¹⁵ progenitors per kilogram of patient body weight, between about 1.5×10⁸ and about 1.5×10¹² progenitors per kilogram of patient body weight, or between about 1×10⁹ and about 5×10¹¹ progenitors per kilogram of patient body weight.

27. The method of claim 24, wherein the therapeutic or prophylactic amount of the subpopulation of enriched progenitors is about 5×10¹⁰ progenitors per kilogram of patient body weight or about 1×10¹⁰ progenitors per kilogram of patient body weight.

28. The method of claim 24, wherein the subpopulation of enriched progenitors is a subpopulation of enriched progenitors of claim 3.

29. The method of claim 24, wherein the subpopulation of enriched progenitors is enriched for mesenchymal progenitors.

30. The method of claim 29, wherein the subpopulation of progenitors is at least about 80% mesenchymal progenitors.

31. The method of claim 29, wherein the subpopulation of progenitors is at least about 90% mesenchymal progenitors.

32. The method of claim 29, wherein the subpopulation of progenitors is at least about 95% mesenchymal progenitors.

33. The method of claim 24, the method further comprising the step of administering islet cells to the patient.

34. The method of claim 33, wherein the islet cells are from the patient being treated or from a donor.

35. The method of claim 34, wherein the islet cells are from a donor cadaver.

36. The method of claim 24, the method further comprising the step of administering capsaicin or substance P to the patient.
